(19) United States
(12) Patent Application Publication
(10) Pub. No.: US 2006/0058686 A1
(43) Pub. Date: Mar. 16, 2006

(54) Method of determining nutritional deficiencies from heart sounds

(76) Inventor: Eric Berg, Alexandria, VA (US)

Correspondence Address: ERIC BERG, 4609 D. PINECREST OFFICE PARK DR, ALEXANDRIA, VA, 22312, US

(21) Appl. No.: 10/939,451

(22) Filed: Sep. 14, 2004

(51) Int. Cl. A61B 5/02

(52) U.S. Cl. 600/483

(57) ABSTRACT

A method of determining nutritional deficiencies from heart sounds by recording heart sound waves, displaying them on paper or a computer screen, and evaluating them to point out certain nutritional deficiencies. A first signal representative of a heart sound is received, then converted into a second signal, which is then filtered, displayed on a graph or monitor, and evaluated to point out nutritional deficiencies.

### BACKGROUND OF THE INVENTION

The present invention generally relates to determining nutritional deficiencies from heart sounds, and more particularly, to determining nutritional deficiencies by recording heart sound waves, displaying them on paper or a computer screen, and evaluating them to point out certain nutritional deficiencies.

Through out research, we have identified a connection between nutritional deficiencies and cardiovascular function. It was found that heart valvular and muscular tone was improved in many cases with specific nutritional implementation. This method involves recording heart sound waves, transmitting them to device which displays them graphically either on paper or computer screen. These graphic representations are then evaluated for nutritional deficiencies. Once the appropriate nutrition is given and consumed a post graph was done to assess changes

### SUMMARY OF INVENTION

Since there is normal parameters on a phoncardiographic (recording heart sounds) heart sound cycle, one can compare pre and post graphs to this normal and see changes in heart tone and valve function. Each part of the graph signifies and correlates to a specific function; for example, if the first sound is too small, the contraction and closing of certain valves is not optimum. When departures from the normal heart sounds have been evaluated, nutrition can be give to restore function to the ideal. It is well documented in the literature that the heart requires certain vitamins and minerals for its function. For example; B1 is essential in the normal anaerobic muscle function of all muscles including the heart. B1 deficiencies show heart muscle weakness. We have observed that even know the heart might not be in a full blown disease state, sub-clinical deficiencies or certain nutrients can alter heart muscle function and be evaluated on phonocardiograph readings.

The purpose of the digital cardiograph is to graphically represent the heart sounds within the cardiac cycle. One of the problems with using a non-digital source stethoscope to listen to sounds is that a portion of heart sounds are below the capacity to hear them. The heart sounds vibrating at 20-40 cycles per second and are not heard by the human ear. By graphing these beats, one can see what is not normally heard through the stethoscope so as to not missing important information.

Blood from the right side of the heart is blue non-oxygenated blood. This blood is pumped into the lungs for oxygenation. Once oxygenated, the red blood now travels back into the heart pump on the left side and is pushed through the artic arch and through the body tissues. The body uses the blood and it is then send back up through the superior and inferior vena cava and back into the right side of the heart. The blood first goes into the atrium. This is a primer pump to get the bigger pump ready. The bigger pump is called the ventricle. The pressure in the left ventricle and aorta are four to five times greater than the right ventricle and pulmonary artery.

The heart differs in that it has a specialize type of muscle called neuromuscular tissue not found anywhere in the body. The uniqueness between the Phonocardiograph and the EKG is that the electro-cardiograph records the electrical activity of the heart, which flows on the top of the ⅓ surface of the heart. The digital endocardiograph records heart sounds, which originate within the heart itself. Each valves graphic representation should look the same.

In the past, it was thought that the sound of the hearts was the valves slapping together. But this has been shown to cause little if not any sound because of the cushioning effect of the blood. Instead, the cause is vibration of the taut valves immediately after closure, as well as vibration of the adjacent blood, walls of the heart, and major vessels around the heart. It is the contraction of the ventricles that cause sudden backflow of blood against the A-V valves, causing them to bulge towards the atria until the chordae tendineae abruptly stop the backbulging. The elastic tautness of the valves then causes the backsurging blood to bounce forward again into each respective ventricle. This sets the blood and the ventricles as well as the valves into vibration and also causes vibrating turbulence in the blood. The vibrations then travel to the chest wall where they can be heard as sound by the stethoscope.

Regarding the placement of the stethoscope, the areas for listening to the different heart sounds are not directly over the valves themselves. The aortic area is upward along the aorta and the pulmonary area is up along the pulmonary artery. The tricuspid area is over the right ventricle, and the mitrial area is over the apex of the heart, which is the portion of the left ventricle nearest the surface of the chest because the heart is rotated so that most of the left ventricle lies behind the right ventricle. This is because the cause of heart sounds are in the ventricles and large arteries.

Regarding rest periods, the diastolic rest period (second rest period) in most normal hearts is twice as long as the systolic rest period at only one pulse rate—72. As the pulse rate increases, the diastolic rest period shortens. The adjustment in pulse rate is made only on the diastolic rest period. The systolic rest period does not change, at least between the pulse rate of 60-90. For each pulse of 4, the diastolic rest period (2nd rest period) changes 1/16th inch or, for each pulse change of 5, the diastolic rest period changes 1/25th second, according to the feed-out speed of 2 inches per second. The relationship allows us to convert any pulse (between 60-90) to 72, at which pulse rate we already had information that the diastolic rest period was twice as long as the systolic rest period. This would indicate coronary artery insufficiency without waiting for the pulse to come to 72. Anything longer in duration would not be abnormal. If this conversion is not done, a doctor could falsely diagnose an abnormal diastolic rest period, when it is perfectly normal.

### BRIEF DESCRIPTION OF THE DRAWINGS

FIG. 1 shows a flow diagram of the invention method.

FIG. 10 shows a view of the first sound.

FIG. 20 shows a view of the second sound.

FIG. 30 shows a view of the cardiac cycle, which includes both sounds.

### DETAILED DESCRIPTION OF THE DRAWINGS

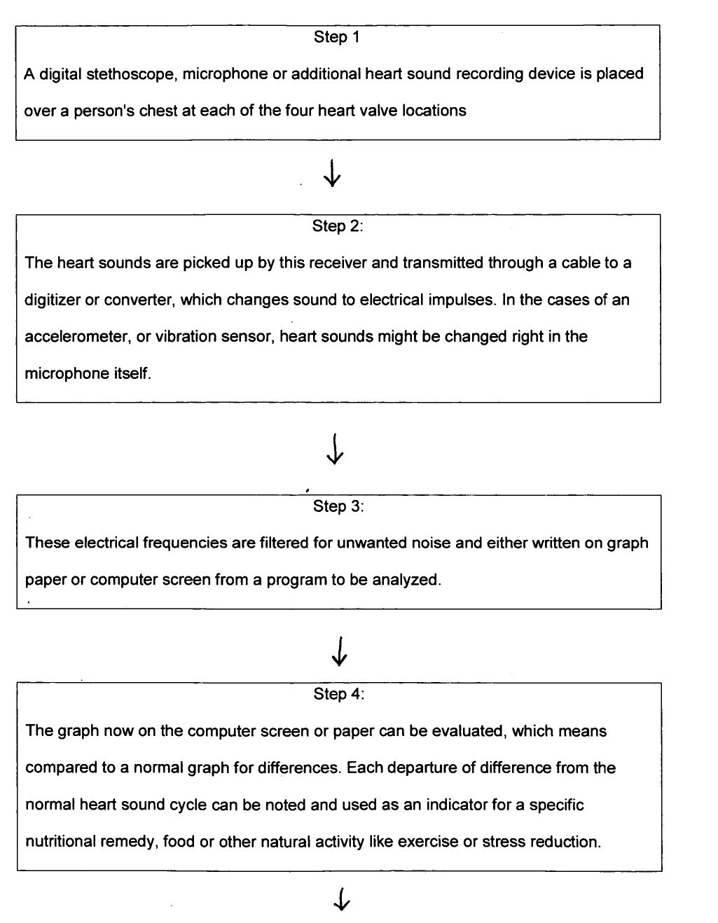

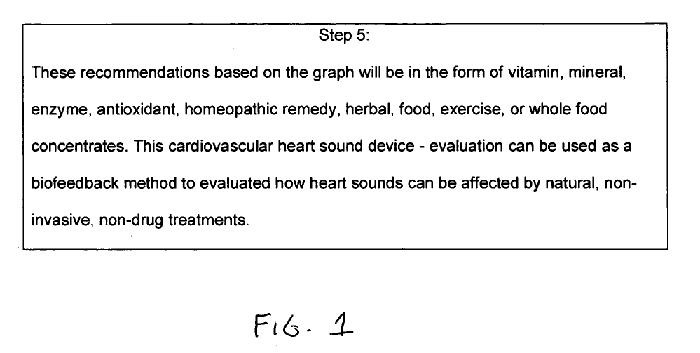

FIG. 1 shows a flow diagram of the invention method. In step 1, a digital stethoscope, microphone or additional heart sound recording device is placed over a person's chest at each of the four heart valve locations. In step 2, the heart sounds are picked up by this receiver and transmitted through a cable to a digitizer or converter, which changes sound to electrical impulses. In the cases of an accelerometer, or vibration sensor, heart sounds might be changed right in the microphone itself. In step 3, these electrical frequencies are filtered for unwanted noise and either written on graph paper or computer screen from a program to be analyzed. In step 4, the graph now on the computer screen or paper can be evaluated, which means compared to a normal graph for differences. Each departure of difference from the normal heart sound cycle can be noted and used as an indicator for a specific nutritional remedy, food or other natural activity like exercise or stress reduction. In step 5, these recommendations based on the graph will be in the form of vitamin, mineral, enzyme, antioxidant, homeopathic remedy, herbal, food, exercise, or whole food concentrates. This cardiovascular heart sound device—evaluation can be used as a biofeedback method to evaluated how heart sounds can be affected by natural, non-invasive, non-drug treatments.

Additionally, under step 5,

1. Heart rate based on the normal range (60-90)

2. 1st and 2nd sound amplitude compared to normal

3. 1st and 2nd sound duration compared to normal

4. 1st and 2nd rest periods for quietness.

5. 2nd sound duration, diastole, converted to pulse of 72

6. Pre-first sound noise (last part of diastolic rest period)-detects stenosis

7. Post-first sound noise (first part of systolic rest period)-detects regurgitation

8. Post-second sound noise (first part of diastolic rest period) regurgitation.

9. If the second part of the 1st heartbeat is deficient. This would indicate a partially decompensated heart muscle because the second part of the 1st beat is of a muscular component.

10. If the second part of the 1st heartbeat is totally missing—This is total decompensated heart muscle. This will only be found on the right side.

11. Extra beats or skipped beats.

12. Doing a comparison of all 4 valve positions with the diastolic rest period duration. Because if there is shortening of the diastolic rest period over the mitral valve—then both coronary arteries are receiving insufficient blood. If the diastolic rest period is deficient over the tricuspid area then only the right coronary blood supply will be efficient.

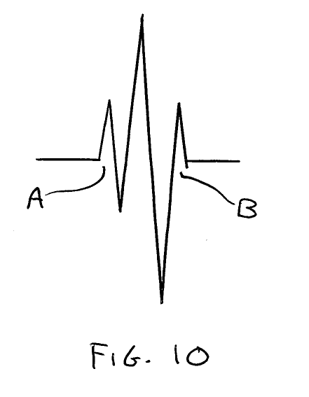

FIG. 10 shows a view of the first sound, which is the closure of the tricuspid (B) and mitral (A) and the opening of the aortic and pulmonary valves. The first sound of the heart beat structures; have less elasticity creating a larger peak on heart sound graphs than the second sound peak. The structures that produce the second sound have more elasticity so they can respond to stretching and stress more easily making a less vibration and a less peak on the graph. The less the elasticity, the lower the vibration. The mass of the blood in the ventricles is much greater than that in the initial part of the great vessels, which means that the force of vibrating mass is also greater. The reason why the second sound is shorter in duration than the first is because the second sound is “dampened” out by the vascular walls much more rapidly than the ventricle walls.

The heart on stethoscope sounds like ‘lub’ and ‘dub’. The “lub” sound is the closure of the A-V valves (tricuspid and mitrial) at the beginning of the contracture (systole) and the opening of the aortic and pulmonary. The loudness of the first sound is almost directly proportional to the rate of change or pressure differences across the valves . . . .

In an abnormal first sound, even though the pressure is different between the right and left side of the heart, the speed of blood flow is the same. If the first sound is faint or absent the following can occur:

1. Myocardial infarction

2. Left bundle branch block

3. Muscular mitrial regurgitation

4. Large aortic regurgitation.

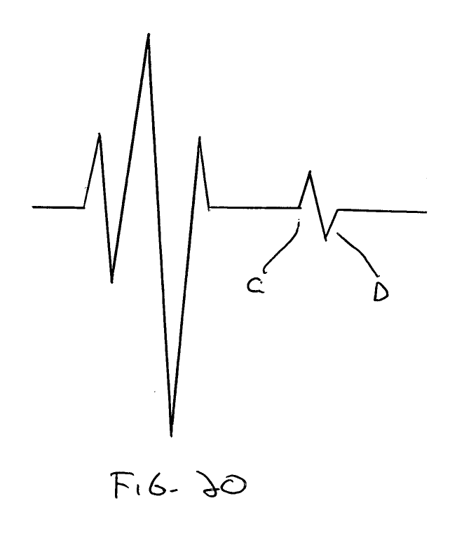

FIG. 20 shows a view of the second sound, which is the closure of the aortic (C) and pulmonary (D) and the opening of the tricuspid and mitral. The second sound results from vibration of the taut, suddenly closed semilunar valves and from vibration of the blood and the walls of the pulmonary artery, the aorta, and, to a much lesser extent, the ventricles. The “dub” is the closure of the semilunar valves (pulmonary and aortic) at the end of the contracture systole. The aortic and pulmonary valve leaflets, which point “upstream” are closed by this reversal of flow, causing the second sound. The valve closure is the result of pressure differential on the side of the valve away from the heart.

The reason the second sound is shorter in duration is because the sound is dampened out by the vascular walls much more rapidly than by the ventricular walls. In the case of the second sound, it is the rate of decrease in ventricular pressure at the end of systole (contraction) that determines the loudness. The magnitude of the rate of decrease is determined mainly by the ventricular systolic pressure at the time the valve closes. In a person with hypertension, the pressure at the time the aortic valve closes may be as great as 200 mm Hg, so the intraventricular pressure falls twice as rapidly as normally, all the way to zero in a few hundredths of second. Therefore the aortic sound is greatly accentuated. Likewise, in pulmonary hypertension, the pulmonary sound is greatly accentuated. On the other hand, in cardiac failure, the second sound diminished to a very low intensity.

In an abnormal second sound, there is excessive resistance in the greater circulation or if the heart ventricles are weak, the reverse surge of blood may strike the valve leaflets with greater forces resulting in a higher-than-normal second sound when recorded in that particular valvular location. A normal ventricle usually keeps this reverse surge under control by relaxing with more or less tone or resistance to this surge providing a sort of cushioning effect, which permits a smooth and normal closure of the aortic and pulmonary valves making it ⅓rd of the height of the first sound. The higher the amplitude of the second sound the weaker the ventricle is presumed to be (or the greater resistance it is encountered in pumping blood into the greater circulation). The heart is required to perform a greater task for its relative strength than should be expected. If the systematic blood pressure is increased, it will cause the heart to pump against greater and greater resistance.

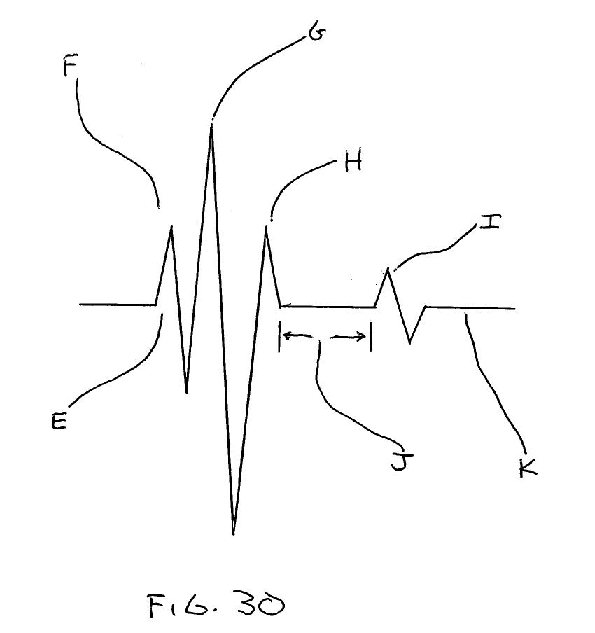

FIG. 30 shows a view of the cardiac cycle, which includes both sounds. It includes: (E) Closure of mitral and tricuspid valves. (A-V valves longer than semi-lunar valves). This is start of systole, 100-140 milliseconds. Apparent opening of the semi-lunar valves happens at the tail end of first sound;

(F)(H) Initial part, 16-20 cycles per second;

(G) Beginning of the ventricular ejection period medial part, 40-50 milliseconds;

(I) Closure of aortic & pulmonary valves (semi-lunar valves, quick snap) Opening of the A-V valves occur at the tail end, which is end of systole

(J) Same pressure between chambers (atrium and ventricles). That time period the contraction of ventricles and the closure of semi-lunar valves. Delay is one of the initial signs of heart deficiency.

(K) Disastole: the interval between closure of semi-lunar and closure of A-V valves.

The entire cardiac cycle requires about 0.85 of a second. S1 lasts for about 0.14 seconds, or 2/25th to 3/25th of a second. S1 has an amplitude of 3× the second sound and two to three its duration. S2 lasts for about 0.11 seconds, or 1/25th of a second. Rest period between first and second sound are 7/25th of a second. Rest period between second and first sound are 14/25th of a second.

Possible deficiencies as they relate to cardiovascular deficiencies may include:

1. Pre and post 81 and 82 noise in the rest periods—murmurs

2. Noise in the rest periods overall—potassium deficiencies

3. Low or absent second sound—calcium and vitamin F deficiencies

4. Decrease first sound (based on the ratio)—B-vitamin or protein deficiency

5. Increased second sound—adrenal stress of hypertension. Vitamin C

6. Increased first sound duration, if over 3/25th of a second—poor or weak ventricle contraction (hypertrophied heart muscle). B Vitamin deficiencies

7. Decrease in duration of the 1st heart beat—dilated heart muscle. This is less than 2/25th of a second. B vitamin deficiencies.

8. Increased second sound duration—thinned blood or anemia (low stomach acid). Acidifiers like apple cider vinegar

9. Decreased diastolic rest period—coronary artery insufficiency, B2, B3 vitamins (these vitamins are vasodilators)

10. Increase heart rate—potassium deficiency as potassium slows the nervous system.

11. Slow pulse rate—phosphorous and B-vitamins which increase heart rate.

12. Extra or missed beats—B vitamin deficiency.

The procedure used to convert the pulse to 72 in a normal graph is as follows:

a. The first sound is 2/2Sth to 3/2Sth of a second in duration.

b. The second sound is ½Sth of a second.

c. The systolic rest period is 1f2 as long as the diastolic rest period.

d. The amplitude of the second sound is ⅓rd the first sound.

e. The first sound is composed of two parts:
i. The first part of the 1st sound is the valvular component.
ii. The second part of the first sound is the muscular component.

2. Ratio of systolic and diastolic should be ⅓ to 2/3 at heart rate of 72

a. The diastolic rest period is synonyms with the blood supply time to the coronary arteries. And this blood supply time must be (at pulse rate of 72) twice as long as the systolic rest period.

b. Patients who have a diastolic rest period longer that twice the systolic period at a pulse rate of 72 are fortunate and would generally indicate an athlete and indicate no danger of Coronary Heart Disease.

c. The patients we will be concerned with are those who have a diastolic rest period with less than twice systolic rest period at a pulse rate of 72.

d. Calculations if the person has a different pulse rate?
i. The change of pulse rate (60-90) does not change the systolic rest period just the diastolic period.
ii. Based on thousands of medical graphs, it has been found that for each pulse change of 4, the diastolic rest period changes 1/16th inch, or each pulse change of 5, the diastolic rest period changes 1/251 second according to the feed-out speed of 2 inches per second on the graph. This relationship data has made it possible to convert any pulse rate to 72. This makes it possible to determine a coronary insufficiency at any pulse rate (60-90) without waiting for a 72 pulse to do this. The measurement will determine the exact amount of coronary deficiency.

e. Recalling that the diastolic rest period is twice as long as the systolic rest period at only one pulse rate (72), it is necessary to convert each cycle to 72 before coronary insufficiency states can be evaluated.

Step 1:

To determine the pulse difference for the cycle being investigated, if the pulse is faster than 72, deduct from the pulse rate. Example: Pulse rate 82, pulse difference (82−72 equals.10). Pulse rate 76, pulse difference (76−72 equals 4). If the pulse is slower than 72, deduct the pulse rate from 72. Example: Pulse rate 67, pulse difference (72−67 equals 5) Pulse rate 60, pulse difference (72−60 equal 12). For each pulse change of 4 the diastolic rest period changes 1/16th inch. Or, for each pulse change of 5, the diastolic rest period changes 1/25th second. That is because 5/16th inches is equivalent to 4/25th second on the graph ruler. Some doctors prefer to use one scale and some the other. An advantage in the 1/16th in scale is the smaller divisions.

Step 2: Divide the pulse difference by 4 (if the 1/16th-inch scale used) or divide the pulse difference by 5 (if the 1/25th second scale used). Example: ( 1/16th-inch sale) Pulse difference 8 divided by 4 equals 2 ( 2/16th inch). Pulse difference 16 divided by 4 equals 4 ( 4/16ths inch) Pulse difference 7 divided by 4 equals 1.75 ( 1.75/16ths inch). Example: ( 1/25th second scale). Pulse difference 10 divided by 5 equals 2 ( 2/25ths second) Pulse difference 7 divided by 5 equals 1.4 ( 1.4/25th second) Pulse difference 13 divided by 5 equals 2.8 ( 2.8/25ths second)

Step 3:

Adjust the diastolic rest period in step 2, using the beginning of the next first sound as the plotting point as follows:

If the original pulse is faster than 72, the diastolic rest period will lengthen as the pulse slows to 72, so the amount computed in step 2 should be added to the diastolic rest period. Place a short vertical line at the adjusting point, measuring forward-from the beginning of the next first sound.

If the original pulse is slower than 72, the diastolic rest period will shorten as the pulse speeds up to 72, so the amount computed in step 2 should be deducted from the diastolic rest period. Place a short vertical line on the chart, measuring back from the beginning of the next first sound.

From the above steps we have determined where the next first sound in the next cycle will start if the pulse were 72. Now it will be necessary to determine where the first sound in the next cycle should start at a 72 pulse rate.

Step 4:

Measure the systolic rest period (from the end of the first sound to the beginning of the second sound). It may be difficult to determine the end of the first sound where murmurs, flutters or tremors or spit sounds exist. By knowing the duration of the normal first sound we can solve this problem by measuring from the beginning of the first sound 2.5/25ths second on the graph (25ths-second scale), and at this point placing a short vertical line.

Step 5:

Multiply the figure determined in step 4 by 2, and from the end of the second sound, measure over on the diastolic rest period, and mark this location by placing a long vertical marker. This locates where the next first sound should start at pulse 72. if irregularities in the graph appear at the second sound, measure from a short line placed ½Sth second to the right of the beginning of the second sound. It will be recalled that ½Sth second is the normal duration of the second sound.

Step 6:

To determine the amount of the coronary insufficiency in 2Sths second per beat, if the short line (from step 3) falls short of the long line (from step 5) use the 25ths second scale on the graph ruler. Measure the distance between them. This measures the number of 25ths second per beat of coronary insufficiency. Mitral for left, and tricuspid for right insufficiency.

Step 7:

To compute the cumulative deficiency per hour, multiply the number of 25ths second deficiency per beat (from step 6) by 3. This gives the number of seconds per minute, or number of minutes per hour cumulative deficiency of coronary insufficiency. Example: 3/25ths second per beat times 3 equal 9 minutes per hour 1.5/25ths second per beat times 3 equals 4.5 minutes per hour. 1/25th second per beat times 3 equals 3 minutes per hour. Recommendations have been made that Aortic and Pulmonary determinations give more accurate coronary insufficiency results than the Mitral and Tricuspid. Often times the graphs are so irregular over the Mitral and Tricuspid areas that it is necessary to use the Aortic and Pulmonary graphs to check against them.

Regarding coronary arteries, the blood in the systolic period on the contraction phase not only forces blood into the system and pulmonary arteries but blood is also forced from the arteries that supply the heart. The diastolic-interval is the relaxation (and filling) phase of the cycle. Not only do the ventricles fill, but also so do the coronary arteries.

Regarding coronary insufficiency, this is a condition in which the need of the myocardium for blood is greater than the amount, which can be delivered. The result is cardiac anoxia and usually severe pains. It may be the result of:

1. Structural lesions of narrowing coronary arteries.

2. Chemical constriction of coronary arteries or abnormal nerve conduction.

3. Very low aortic diastolic pressure

4. When blood is poorly saturated with oxygen

5. Low Hemoglobin.

Regarding murmers, a murmur consists of a deviation from the normal of the usual straight line on the graph. It doesn't look like the teeth of a saw. It always has a waveform, like the top silhouette of Hollywood Hills. Sharp wave forms are the latter part of an asynchronous closure. They may be decompensated waves (lazy waves) that didn't reach their full amplitude. A murmur is always revealed as a waveform. The graph has a straight line between the sounds, and murmurs occur closer to the sounds, but the base line is straight.

These sounds are made by the vibration of:

1. Valve closure (sections of valve leafs). It is not the slapping the valves closing is the vibration of the taut valves immediately after closure, as well as the vibration of the adjacent blood, walls of the heart, and the major vessels around the heart. In the generation of the 1st sound, contraction of the ventricles causes sudden backflow of blood against the A-V valves, causing them to bulge toward the atria until the chordae tendineae abruptly stop the backbulging. The elastic tautness of the valves then cause the backsurging blood to bounce forward again into each ventricle. This sets the blood and ventricles and valves into vibration. The second sound comes from the sudden taut vibration of the closing of the semi-lunar valves, and blood as well as the walls of the pulmonary artery and aorta.

2. Sudden acceleration and deceleration of blood (pressure differences) through heart wall chambers and arteries.

3. The blood contents rebounding off the elastic heart wall structures.

You do not hear the opening of valves. This is a relatively slow process that makes no Noise. This is compared to a mouth (opening), the air inside or fluid, with the opening and closing of the lips (valves) from tension (pressure) produced by the inside walls.

The pressure changes in the left ventricle and the aorta are 4-5× greater than the right ventricle and pulmonary artery. However, the accelerations and decelerations in each on of these sectors have the same relationship.

The first sound sound depends fundamentally on the left ventricle and aorta, except in a hyper kinetic heart or in a right ventricular hypertension. This is why the first heart sound is faint or absent in several conditions in which there is a diseased left ventricle and health A-V valve and right ventricle (myocardial infarction, left bundle branch block, muscular mitral and aortic regurgitation). Increased 2^{nd} sound means right ventricular enlargement with pulmonary hypertension.

Murmurs:

1. Accumulation of cholesterol, calcium or scar tissue on valves.

2. Weakened heart muscles.

3. Murmurs occur over their position.

4. They are also known as stenosis or constriction.

5. May indicate regurgitation of blood because of a poorly closed valve.

Many murmers are produced by an increase in the velocity of blood flow, or by a change in the viscosity of blood flow, from impediments placed in its way and from a sudden contracture or dilation of the tube through which the blood flows. As the heart progressively weakens the velocity of the blood is decreased and murmurs no longer are produced. Stenosis-makes a murmurs sound before beat. Regurgitation-makes a sound after the first beat. In no indication of presystolic, then stenosis is not the cause. It may be:

1. Endocarditis: Inflammation inside the lining of the heart.

2. Dialation of ventricles: Lack of tone; it will always have an accentuated relative amplitude of the second sound but the duration generally is short.

3. Hypertension: If hypertension has been present the mitral leak is a “compensatory” reaction (safety valve). Anemia may be present.

Regarding heart muscle involvement, this condition is shown by the relative amplitude and duration of the first and second. This is because these sounds are caused by the closure of the valves and in turn, the valve closure are the result of the build-up of pressure differential by the constriction or relaxation of one or more of the heart muscles.

Regarding indicated weak heart muscle, this shows up as reduced amplitude of the first sound and increased amplitude of the second.

a. This is because the ventricle does not contract suddenly enough to snap the mitral and tricuspid valve shut and produce a loud first sound.

b. Also the relaxation of the ventricle is so sudden that the aortic and pulmonary valves snap shut.

c. This condition is common; not relative serious per se and generally indicates a dilated (enlarged) heart on either left or right side depending upon the location of the graph.

d. It may also indicate high blood pressure. This is the only method of diagnosing hypertension in the pulmonary circulation.

As this condition becomes worse, the first sound increases in duration.

a. This is because it takes longer for the ventricle to shut the mitral and tricuspid valves. (This is generally seen in cases of coronary trouble). The diastolic rest period is also an indicator of coronary circulation.

As this condition becomes still worse the amplitude of the first sound becomes still less and then the duration is cut to less than normal.

a. This is because the ventricle no longer can competently shut the mitral and tricuspid valves and is a very serious picture.

Regarding placement of different heart sounds, with the stethoscope placed over anyone of the valves locations, the sounds from all the other valves can also be heard. One evaluates each sound from each valve location noting the changes in each. The location of the stethoscope is not directly over the valves themselves. The aortic area is upward along the aorta, the pulmonary is upward along the pulmonary artery, the tricuspid area is over the right ventricle, and the mitral area is over the apex of the heart, which is the portion of the left ventricle nearest the chest because the heart is rotated so that most of the ventricle lies behind the right ventricle. Sounds from A-V valves (mitral and tricuspid) are transmitted to the chest wall through each ventricle. Sounds from the semi-lunar valves are transmitted along the great vessels.

I claim:

1. A method of determining nutritional deficiencies from heart sounds, comprising:
Receiving a first signal representative of a heart sound;
Converting the first signal to a second signal;
Filtering the second signal;
Displaying the filtered signal; and
Evaluating the displayed signal:

2. The method of claim 1, wherein converting the first signal includes digitizing the first signal.

3. The method of claim 1, wherein filtering the second signal includes passing frequencies between x and y.

4. The method of claim 1, wherein displaying the filtered signal includes displaying the filtered signal in a monitor.

5. The method of claim 1, wherein evaluating the displayed signal includes analyzing the heart rate, amplitude, duration, rest periods, diatole, stenosis, regurgitation, decompensated heart muscle, extra or skipped beats, or all four valve positions.
The above claims may also include other analysis, and are not limited to the mentioned items.
